(19) United States
(12) Patent Application Publication
(10) Pub. No.: US 2022/0164011 A1
(43) Pub. Date: May 26, 2022

(54) SYSTEMS AND METHODS FOR DYNAMIC ELECTRONIC DEVICE TEMPERATURE THRESHOLD ADJUSTMENT

(71) Applicant: Facebook Technologies, LLC, Menlo Park, CA (US)

(72) Inventors: Vivek Sahu, Redmond, WA (US); Alex Ockfen, Bothell, WA (US); Janet Rose Griffin, Woodinville, WA (US)

(21) Appl. No.: 16/953,883

(22) Filed: Nov. 20, 2020

(51) Int. Cl. G06F 1/20; G05B 19/4155; G01K 3/00; G01K 11/12; A61B 5/024; A61B 5/08

(52) U.S. Cl. CPC G06F 1/206; G05B 19/4155; G01K 3/005; G05B 2219/49219; A61B 5/024; A61B 5/0816; G01K 11/12

(57) ABSTRACT

The disclosed computer-implemented method may include (1) obtaining, within a local environment in which a user is in physical contact with an electronic device, a current value for each of a plurality of measurable characteristics associated with at least one of the user or the local environment, (2) determining, based on the current value for each of the plurality of measurable characteristics, a temperature threshold for the electronic device, (3) measuring a current temperature of the electronic device, (4) comparing the current temperature to the temperature threshold, and (5) initiating, in response to the current temperature exceeding the temperature threshold, a heat mitigation operation of the electronic device to lower the current temperature. Various other methods, electronic devices, and computer-readable media are also disclosed.

### BRIEF DESCRIPTION OF THE DRAWINGS

The accompanying drawings illustrate a number of exemplary embodiments and are a part of the specification. Together with the following description, these drawings demonstrate and explain various principles of the present disclosure.

FIG. 1 is a block diagram of an exemplary operating environment in which various methods and devices, as described herein, may be employed.

FIG. 2 is a flow diagram of an exemplary method for dynamic temperature threshold adjustment of an electronic device.

FIG. 3 is a block diagram of an exemplary electronic device in which the method of FIG. 2 may be employed.

FIG. 4 is a flow diagram of another exemplary method for dynamic temperature threshold adjustment of an electronic device.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the present disclosure covers all modifications, equivalents, and alternatives falling within the scope of the appended claims.

### DETAILED DESCRIPTION OF EXEMPLARY EMBODIMENTS

In many types of electronic devices, such as desktop and laptop computers, smartphones, and the like, a predefined device temperature threshold may be used to initiate one or more heat mitigation operations, such as reducing a speed at which electronic circuitry is operating within the device, to reduce the temperature of the device. In some cases, the temperature threshold may be set based on whether the device may experience temporary errors or permanent damage at temperatures exceeding the threshold.

The present disclosure is generally directed to dynamically adjusting a device temperature threshold (e.g., in real-time) based on a plurality of measurable characteristics, such as those associated with the device, the surrounding environment, and/or the user. As will be explained in greater detail below, embodiments of the present disclosure may employ characteristics that affect the user's perception of heat being received from the device in some manner to adjust the temperature threshold. In some examples, the measured characteristics may include, but are not limited to, environmental conditions, user physiological parameters, device conditions, and/or user-specific characteristics.

Features from any of the embodiments described herein may be used in combination with one another in accordance with the general principles described herein. These and other embodiments, features, and advantages will be more fully understood upon reading the following detailed description in conjunction with the accompanying drawings and claims.

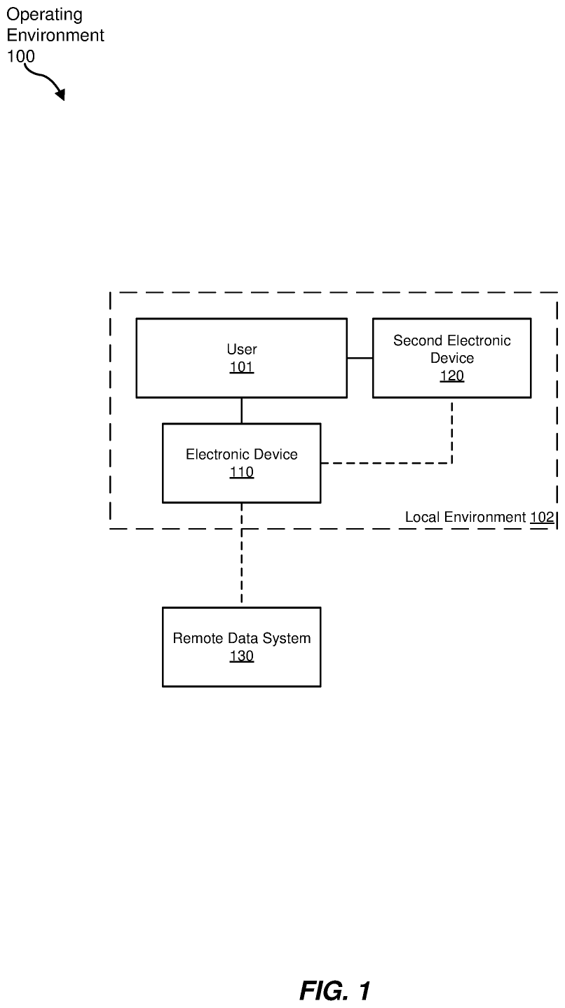

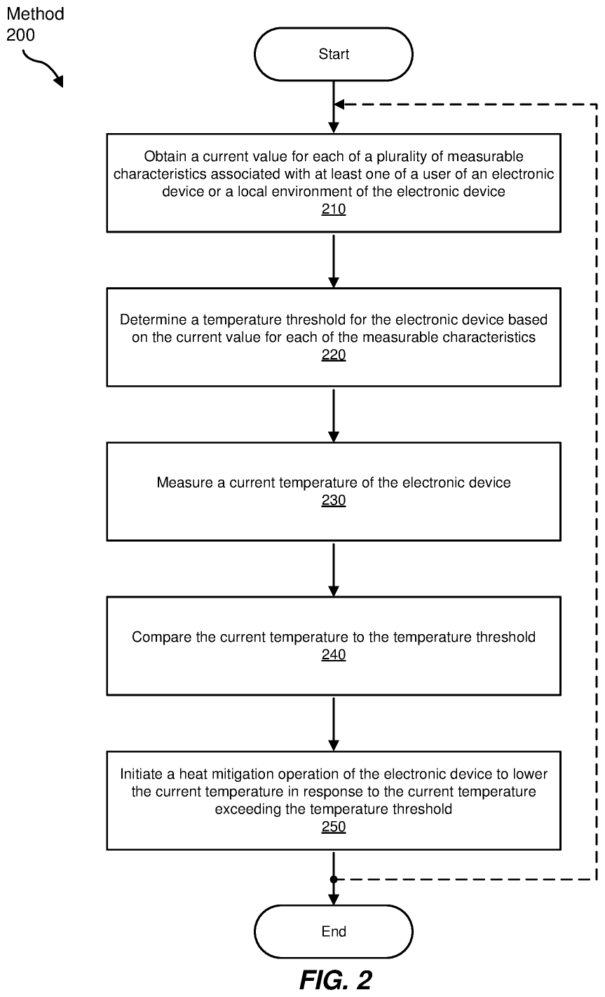

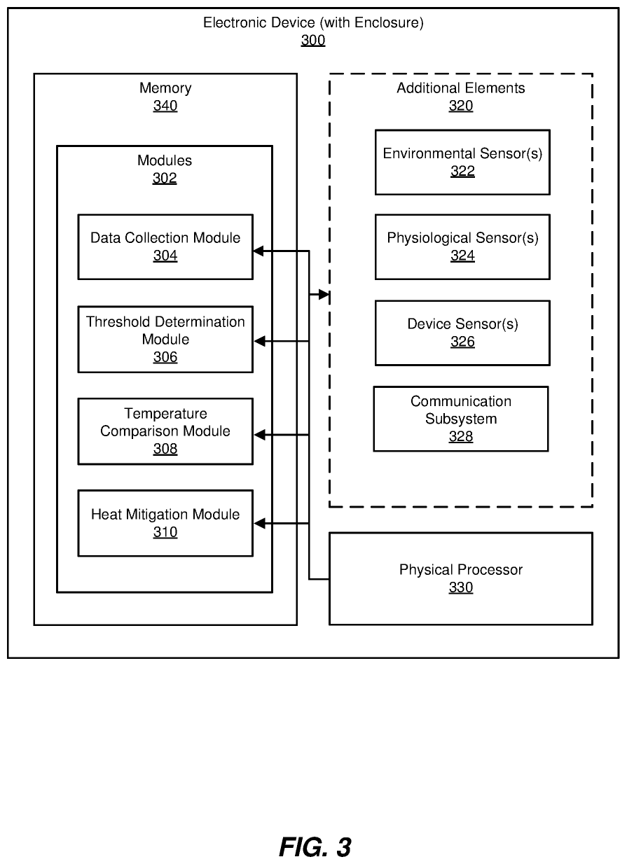

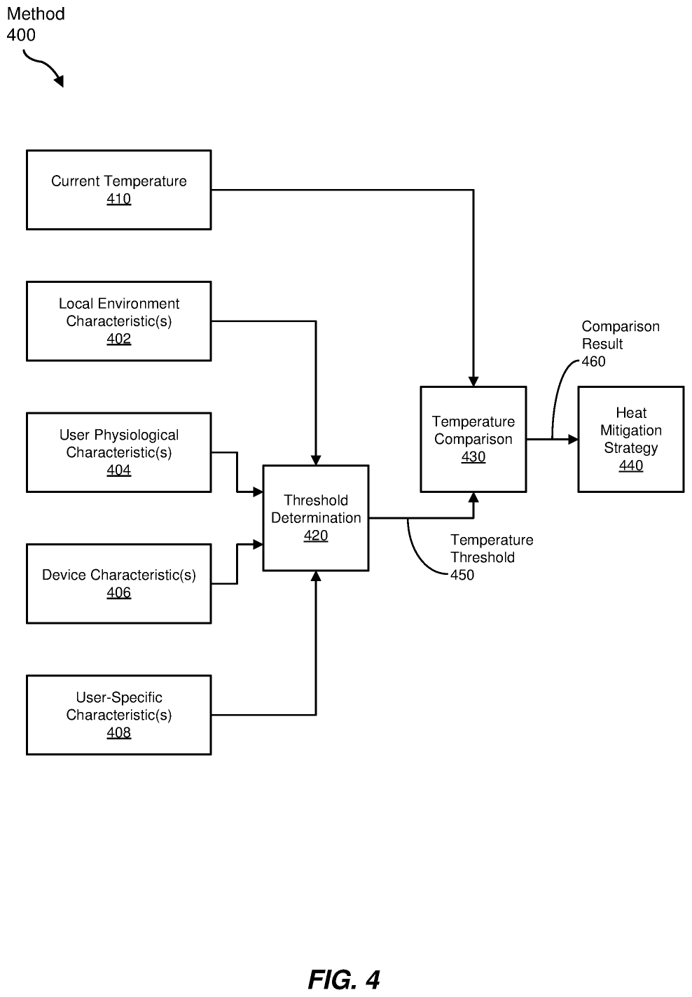

The following will provide, with reference to FIGS. 1-4, detailed descriptions of methods and associated devices and systems for dynamically adjusting a device temperature threshold based on a plurality of measurable characteristics. An exemplary operating environment in which such methods and devices may be employed is discussed in conjunction with FIG. 1. In connection with FIG. 2, an exemplary method for dynamic temperature threshold adjustment of an electronic device is described. An exemplary electronic device that may employ such a method is discussed in relation to FIG. 3. Additionally, in connection with FIG. 4, another exemplary method for dynamic temperature threshold adjustment of an electronic device is described.

FIG. 1 is a block diagram of an exemplary operating environment 100 in which various methods and devices, as described herein, may be employed. In operating environment 100, a user 101 may be employing an electronic device 110 to perform one or more tasks. Examples of electronic device 110 may include, but are not limited to, a smartphone, a smartwatch, a tablet computer, an artificial reality device (e.g., a virtual reality (VR) head-mounted device (HMD), augmented reality glasses, or the like), a laptop or desktop computer, a gaming system, and so on. In some examples, electronic device 110 may be coupled with user 101, such as being handheld by user 101 (e.g., in the case of a smartphone, tablet, or handheld gaming system), strapped to or otherwise in contact with user 101 (e.g., in the case of a smartwatch, an HMD, a pair of glasses, a laptop computer, and so on), or at least in close proximity to user 101 (e.g., in the case of a desktop computer). Other examples of electronic device 110 are also possible.

As depicted in FIG. 1, user 101 and electronic device 110 may be located within a local environment 102. In some embodiments, local environment 102 may be defined as any localized space or area encompassing both user 101 and electronic device 110 (e.g., such that at least some ambient conditions, such as temperature, light, wind, humidity, and/or other conditions, may be attributable to both user 101 and electronic device 110). In some examples, local environment 102 may be defined as a particular room or other indoor space in which user 101 and electronic device 110 are located, or a generalized outdoor area (e.g., identifiable via Global Positioning System (GPS) location data) where user 101 and electronic device 110 are located. In some embodiments, electronic device 110 may measure one or more characteristics associated with electronic device 110, user 101, or local environment 102 that may be utilized to dynamically determine a temperature threshold for electronic device 110.

Further, as illustrated in FIG. 1, electronic device 110 may be in communication with a second electronic device 120 that may also be coupled to user 101 in some fashion. For example, presuming electronic device 110 is a smartphone held by user 101, second electronic device 120 may be a smartwatch or fitness device worn on a wrist of user 101. In such cases, second electronic device 120 may provide the one or more measurable characteristics associated with at least user 101 or local environment 102 to electronic device 110 for determining the temperature threshold. In some examples, communication between electronic device 110 and second electronic device 120 may be facilitated by way of a WiFi connection, a Bluetooth® connection, a wired connection, and so on.

Additionally, electronic device 110 may be communicatively coupled with a remote data system 130 located outside local environment 102 such that remote data system 130 may provide one or more of the measurable characteristics associated with user 101, electronic device 110, or local environment 102. In some cases, remote data system 130 may be a remote data server accessible via the Internet. Such measurable characteristics may include, but are not limited to, weather information associated with local environment 102, user 101 profile information, and the like.

More specifically, in some embodiments, the plurality of characteristics mentioned above (e.g., as measured by electronic device 110 and/or second electronic device 120) may include measurable parameters or conditions associated with local environment 102, such as ambient light intensity and/or wavelength spectrum, wind speed, humidity, and so on. Further, such characteristics may include measurable dynamic parameters associated with user 101, such as heart rate, respiration, perspiration, metabolic activity, physical activity, and so on.

Also, in some examples, the plurality of characteristics may include user profile information (e.g., information indicating whether user 101 is particularly deterred from use of electronic device 110 at higher-than-normal operating temperatures, medical conditions experienced by user 101 that may affect the perception of heat by user 101, actions taken by user 101 (e.g., stopping a video stream, putting down electronic device 110, etc.) in the presence of previous periods of elevated heat generated by electronic device 110, historical and/or current duration of usage of electronic device 110, historical and/or current types of usage of electronic device 110 by user 101 (e.g., reading of electronic books, listening to podcasts or audio tracks, streaming of video clips, capturing video, and so on).

Further, the plurality of characteristics may include characteristics of electronic device 110 (e.g., a size or location of an area of electronic device 110 likely to be in contact with user 101, heat transfer characteristics (e.g., heat flux) or other thermal characteristics of electronic device 110, a charge level or charge usage rate of a battery in electronic device 110, and the like).

FIG. 2 is a flow diagram of an exemplary computer-implemented method 200 for dynamic adjustment of a temperature threshold for an electronic device (e.g., electronic device 110 of FIG. 1). The steps shown in FIG. 2 may be performed by any suitable computer-executable code, including the device(s) illustrated in FIG. 1. In one example, each of the steps shown in FIG. 2 may represent an algorithm whose structure includes and/or is represented by multiple sub-steps, examples of which will be provided in greater detail below.

In the method 200 of FIG. 2, at step 210, a current value may be obtained for each of a plurality of measurable characteristics associated with at least one of a user (e.g., user 101 of FIG. 1) of the electronic device and a local environment (e.g., local environment 102 of FIG. 1) of the electronic device. In some embodiments, the measurable characteristics may include, but are not limited to, user-specific parameters such as current physiological parameters of the user, activity level of the user, and so on, and parameters associated with the local environment such as ambient light, wind speed, humidity, and the like. As described in greater detail below, in other examples, characteristics of the electronic device, types and durations of operations or use cases currently performed by the device (e.g., playing of audio and/or video, capturing of audio and/or video, transmission and reception of data via cellular network or Wi-Fi, etc.), and so on may be measured as well. In at least some examples, the plurality of measurable characteristics may be obtained and/or updated repeatedly (e.g., periodically, continually, or in real-time).

At step 220, a temperature threshold for the electronic device may be determined based on the current value for each of the measurable characteristics. In some embodiments, the temperature threshold may be a mathematical combination (e.g., a weighted combination) of the measurable characteristics, a value referenced or indexed in a data structure (e.g., a lookup table) using the measurable characteristics, or by some other process. Further, in some examples, the temperature threshold may be determined or updated repeatedly or continually (e.g., in real-time) based on updated values of the measurable characteristics being employed to determine the threshold.

Further, in some examples, the temperature threshold may be adjusted or at least partially based on one or more static characteristics of the electronic device that may affect a measured temperature of the electronic device. Such characteristics may include, but are not limited to, a location of one or more temperature sensors located in or on the electronic device, an indication of the amount of heat (e.g., as measured by the one or more temperature sensors) that may be transferred to the user from the electronic device, the location and/or amount of surface area on the electronic device that is available to be contacted by the user, and so on.

At step 230, a current temperature of the electronic device may be measured. In some embodiments, as mentioned above, one or more temperature sensors may be employed to measure the temperature at corresponding locations in or on the electronic device. In some embodiments, the measured temperature associated with each of multiple temperature sensors may be averaged, summed in a weighted manner, or otherwise combined to determine a current temperature for the electronic device.

At step 240, the current temperature of the electronic device may be compared to the current temperature threshold. Further, at step 250, in response to the current temperature exceeding the temperature threshold, a heat mitigation operation of the electronic device may be initiated to lower the current temperature. In some embodiments, the heat mitigation operation may include reducing an operational speed or capacity of the electronic device, such as a reduced processor speed, a reduced communication data rate, and the like. Additionally or alternatively, the heat mitigation operation may include disabling one or more electronic circuits or functions within the electronic device, such as a wireless communication transceiver, an audio/video processor, and the like. In yet other examples, the heat mitigation operation may include mechanical heat reduction techniques, such as increased fan speeds, activation of previously inactive fans, and so on to reduce the current temperature of the electronic device.

In at least some embodiments, as indicated above, steps 210-250 may be performed continually or repetitively (e.g., in real-time) while the user employs the electronic device, as indicated by the dashed line in FIG. 2. Consequently, in some examples, the temperature threshold may be updated from time to time as measurable characteristics associated with the user (e.g., user heart rate, user respiration, and the like), the local environment (e.g., ambient light level and/or type, temperature, humidity, and so on) change over time.

FIG. 3 is a block diagram of an exemplary electronic device 300 that may employ dynamic adjustment of a temperature threshold based on a plurality of measurable characteristics, such as those associated with electronic device 300 (e.g., serving as electronic device 110 of FIG. 1), a surrounding local environment (e.g., local environment 102 of FIG. 1), and/or a user of electronic device 300 (e.g., user 101 of FIG. 1). Electronic device 300, in some embodiments, may be partially or entirely enveloped by an enclosure with which the user may establish and maintain physical contact while using electronic device 300. In other examples, the user may be within some distance of the enclosure such that heat from electronic device 300 may still be experienced by the user. Examples of electronic device 300 may include, but are not limited to, a smartphone, a smartwatch, a tablet computer, an artificial reality device (e.g., a VR HMD, augmented reality glasses, or the like), a laptop or desktop computer, a gaming system, and so on.

As illustrated in FIG. 3, electronic device 300 may include one or more modules 302 for performing one or more tasks. As will be explained in greater detail below, modules 302 may include a data collection module 304, a threshold determination module 306, a temperature comparison module 308, and a heat mitigation module 310. Other modules 302 may be included to perform various operations associated with electronic device 300, but such modules 302 are not discussed further herein to simplify and focus the following discussion.

In certain embodiments, one or more of modules 302 in FIG. 3 may represent one or more software applications or programs that, when executed by electronic device 300, may cause electronic device 300 to perform one or more tasks. In other examples, one or more of modules 302 may represent modules stored and configured to run on one or more computing devices other than electronic device 300.

As illustrated in FIG. 3, electronic device 300 may also include one or more memory devices, such as memory 340. Memory 340 generally represents any type or form of volatile or non-volatile storage device or medium capable of storing data and/or computer-readable instructions.

As also depicted in FIG. 3, electronic device 300 may further include one or more physical processors, such as physical processor 330. Physical processor 330 generally represents any type or form of hardware-implemented processing unit capable of interpreting and/or executing computer-readable instructions. In one example, physical processor 330 may access and/or modify one or more of modules 302 stored in memory 340. Additionally or alternatively, physical processor 330 may execute one or more of modules 302 to dynamically adjust a temperature threshold based on a plurality of measurable characteristics that may change over time, such as what may be used to control heat generation by electronic device 300.

As illustrated in FIG. 3, electronic device 300 may also include one or more system hardware 320 components, such as one or more environmental sensors 322, one or more physiological sensors 324, one or more device sensors 326 (e.g., one or more temperature sensors), and/or a communication subsystem 328. In some embodiments, an environmental sensor 322 may be any sensor that detects or measures some characteristic or aspect of the local environment (e.g., location, ambient light level and/or wavelength spectrum, humidity, wind speed, and the like), while a physiological sensor 324 may be any sensor that detects or measures a physiological characteristic or aspect of the user (e.g., heart rate, respiration, perspiration, skin temperature, and so on). Examples of environmental sensors 322 and/or physiological sensors 324 may include, but are not limited to, a visible light camera, an infrared camera, an ambient light sensor (ALS), an inertial measurement unit (IMU), a proximity sensor (e.g., for detecting a proximity of the user to electronic device 300), and a heat flux sensor. In some examples, a sensor may serve as both an environmental sensor 322 and a physiological sensor 324. While environmental sensors 322 and physiological sensors 324 are described herein as residing within electronic device 300, in other embodiments, one or more environmental sensors 322 and/or physiological sensors 324 may be located external to electronic device 300 but within the local environment, such as in a separate electronic device associated with the user (e.g., within second electronic device 120 of FIG. 1).

One or more device sensors 326 may also be included in electronic device 300, such as one or more temperature sensors to measure the current temperature at one or more corresponding places in or on electronic device 300 (e.g., within the enclosure, at an external surface of the enclosure, etc.). In some embodiments, multiple temperature sensors may facilitate a more detailed understanding of the distribution of heat of electronic device 300 and/or an identification of one or more components within electronic device 300 most responsible for generating that heat, which may be employed to determine a particular heat mitigation operation or strategy to employ. Other device sensors 326 may include sensors for charge level or usage rate of a battery in electronic device 300, sensors for detecting user contact with electronic device 300, and so on.

Communication subsystem 328 (e.g., a wireless communication subsystem, such as a cellular communication subsystem, a Bluetooth® communication subsystem, a WiFi communication subsystem, or the like), in some embodiments, may receive data representing one or more of the plurality of measurable characteristics employed to determine a temperature threshold for electronic device 300. For example, communication subsystem 328 may receive data representing physiological characteristics of the user (e.g., heart rate) from a second electronic device (e.g., a fitness watch) worn by the user. In other examples, communication subsystem 328 may receive data representing characteristics of the local environment (e.g., weather-related data, such as temperature, humidity, wind speed, cloud coverage, and the like) from another electronic device within the local environment, or from a communication device (e.g., a data server) outside the local environment, such as via a weather application executing on electronic device 300. Additionally, in some embodiments, communication subsystem 328 may receive other data employed in the determination of the temperature threshold for electronic device 300 (e.g., user profile data) that is not measured in real-time.

Returning to modules 302, data collection module 304 may receive data or values of the plurality of measurable characteristics of the user, the local environment, or electronic device 300 (e.g., from environmental sensors 322, physiological sensors 324, and/or communication subsystem 328), one or more current temperature values of electronic device 300 (e.g., from device sensors 326), and other data (e.g., user profile information) that may affect the temperature threshold (e.g., from communication subsystem 328, from memory 340, and/or elsewhere). As mentioned above, data collection module 304 may repeatedly or continually collect the measurable characteristics and the current temperature values for availability to threshold determination module 306 and temperature comparison module 308 to continually determine whether electronic device 300 should perform a heat mitigation operation.

In some embodiments, threshold determination module 306 may calculate or otherwise determine (e.g., via mathematical calculation (e.g., a weighted combination of values), via accessing a stored data structure (e.g., a lookup table), and/or other means) the temperature threshold using the measurements and other data collected by data collection module 304. Temperature comparison module 308 may then compare the temperature threshold to one or more values, or some combination thereof, of the one or more measured temperature values of electronic device 300.

Heat mitigation module 310, in some embodiments, may initiate or continue a heat mitigation operation to lower a current temperature of electronic device 300 (e.g. in response to one or more current temperature values of electronic device 300 exceeding a temperature threshold). In some examples, heat mitigation module 310 may select from more than one type of heat mitigation strategy (e.g., control of a heat reduction fan, a reduction in operating speed of one or more components of electronic device 300, a shutdown of one or more components, and the like). Further, in some examples, heat mitigation module 310 may base its selection of a heat mitigation strategy on a current use of electronic device 300, previous feedback from the user on prior experiences of the user with electronic device 300, and so on.

FIG. 4 is a flow diagram of another exemplary method 400 for dynamic temperature threshold adjustment of an electronic device. In some embodiments, method 400 may be performed by electronic device 110, electronic device 300, or some other electronic device or devices not specifically disclosed herein.

In method 400, one or more types of data may be received by threshold determination function 420 to determine a temperature threshold for an electronic device (e.g., electronic device 300). In some embodiments, the input data may include one or more local environment characteristics 402, user physiological characteristics 404, device characteristics 406, and or user-specific characteristics 408. Local environment characteristics 402 may include, for example, a light intensity and/or wavelength spectrum, an ambient temperature, a humidity, and/or a wind speed prevailing in the local environment of the electronics device. User physiological characteristics 404 may include, for example, heart rate, respiration rate and/or depth, metabolic activity, and/or physical activity of the user. Device characteristics 406 of the electronic device may include, for example, device contact area (e.g., area location and/or size of the electronic device that typically may be physically contacted by the user, or that is currently being contacted by the user), current duration of usage of the electronic device by the user, a heat flux of the device, a battery charge level or battery charge usage rate, and so on. User-specific characteristics 408 of the user may include dynamic and/or static characteristics describing some aspect of the user. Examples of dynamic user-specific characteristics 408 may include heart rate, respiration rate and/or depth, perspiration, level of physical activity, and/or level of metabolic activity, while static user-specific characteristics may include demographics (e.g., age, gender, ethnicity, and the like), medical conditions (e.g., a medical condition that results in an above-average sensitivity to heat), user expectations regarding tradeoffs in device performance level versus desired comfort level, and so on. At least some of this input data to threshold determination function 420 may be updated and provided continually to threshold determination function 420, thus allowing threshold determination function 420 to determine a temperature threshold 450 dynamically for the electronic device.

Threshold determination function 420 may employ the input data it receives to generate a dynamic temperature threshold 450 for the electronic device to improve or maximize the user's experience in using the electronic device. In some cases, temperature threshold 450 may be maintained to be less than some operating temperature at which decreased performance or physical impairment of the electronic device may result. Also, in some examples, temperature threshold 450 may be set such that the user remains comfortable during use of the electronic device while providing at least an acceptable level of performance. In some embodiments, user-specific characteristics 408 may include data indicating the user's relative willingness to withstand some discomfort (e.g., an increased amount of heat generated by the electronic device) for an increased level or lengthened duration of performance. Further, in some examples, the user's willingness may be related to the particular device functions or use cases the user is currently employing. For example, the user may be willing to experience a higher level of heat from the device when streaming video compared to reading a document using the electronic device. In another example, the user may be willing to experience a higher level of heat when capturing video compared to streaming (viewing) video or listening to audio, as capturing video may be viewed as a higher priority use case than other possible use cases of the device employable by the user.

Moreover, in some examples, the experience of the user may be affected by various factors associated with the local environment (e.g., as reflected by local environment characteristics 402), such as whether the user is indoors in an air-conditioned environment, indoors in a non-air-conditioned environment, outdoors on a sunny day, outdoors on a cloudy day, and the like, which may affect the user's perception of the heat generated by the device. For example, whether the device is located outdoors on a sunny or cloudy day, or located indoors, may be determined at least in part by output from an ALS that indicates a level and/or bandwidth spectrum of light in the local environment. More specifically, a first threshold may be set in the case that the device is located outdoors, and a second threshold may be set that is higher than the first threshold when the device is located in an air-conditioned indoor environment.

In some embodiments, some inputs to threshold determination function 420 may be interpreted to determine whether a user is making physical contact, or is in close proximity, with the electronic device. For examples, capacitive sensors, IMUs, and the like may detect contact with, or movement of, the device, indicating that the user may be grasping the device. Accordingly, if the user is not in contact with the device, temperature threshold 450 may be increased due to a reduced effect of the heat generated by the electronic device on the user. Similarly, in examples in which the user may physically interact with the electronic device in various ways (e.g., held by hand, worn on the wrist using a strap, and so on), threshold temperature may be adjusted based on the heat sensitivity of the particular portion of the user making contact with the electronic device.

As further shown in FIG. 4, temperature comparison function 430 may then compare the current temperature threshold 450 with one or more current temperature 410 values, yielding a comparison result 460 that is provided to a heat mitigation strategy function 440 to indicate whether current temperature 410 exceeds temperature threshold 450, thus possibly causing a particular heat mitigation operation to be initiated or continued. In some embodiments, each of a plurality of current temperatures 410 may correspond with a particular location (e.g., a particular functional portion) of the electronic device, thus indicating whether that portion is generating excessive heat. Accordingly, in some examples, a heat mitigation operation (e.g., reduction of operating speed, termination of one or more functions, increased airflow, etc.) may be directed to the particular portion of the electronic device causing or contributing to the excessive heat.

In view of the various embodiments described above in conjunction with FIGS. 1-4, a temperature threshold for an electronic device may be dynamically adjusted over time in response to changes in user physiological factors, aspects of the surrounding environment, a status of the electronic device itself, and other factors. Consequently, in some embodiments, user satisfaction with the electronic device may be enhanced by maximizing operational capability of the device while maintaining a temperature for the device that is comfortable for the user under the current circumstances.

### EXAMPLE EMBODIMENTS

Example 1: A computer-implemented method for dynamic temperature threshold adjustment for an electronic device may include (1) obtaining, within a local environment in which a user is in physical contact with the electronic device, a current value for each of a plurality of measurable characteristics associated with at least one of the user or the local environment, (2) determining, based on the current value for each of the plurality of measurable characteristics, a temperature threshold for the electronic device, (3) measuring a current temperature of the electronic device, (4) comparing the current temperature to the temperature threshold, and (5) initiating, in response to the current temperature exceeding the temperature threshold, a heat mitigation operation of the electronic device to lower the current temperature.

Example 2: The computer-implemented method of Example 1, where the plurality of measurable characteristics may include a characteristic of light detected within the local environment.

Example 3: The computer-implemented method of Example 2, where the characteristic of the light detected within the local environment may include at least one of an intensity of the light or a wavelength spectrum of the light.

Example 4: The computer-implemented method of any one of Examples 1-3, where the plurality of measurable characteristics may include a wind speed detected within the local environment.

Example 5: The computer-implemented method of any one of Examples 1-3, where the plurality of measurable characteristics may include a humidity detected within the local environment.

Example 6: The computer-implemented method of any one of Examples 1-3, where the plurality of measurable characteristics may include an ambient temperature detected within the local environment.

Example 7: The computer-implemented method of any one of Examples 1-3, where the plurality of measurable characteristics may include a heart rate of the user.

Example 8: The computer-implemented method of any one of Examples 1-3, where the plurality of measurable characteristics may include a respiration characteristic of the user.

Example 9: The computer-implemented method of any one of Examples 1-3, where obtaining the current value for each of the plurality of measurable characteristics may include measuring the current value for at least one of the plurality of measurable characteristics using a sensor of the electronic device.

Example 10: The computer-implemented method of any one of Examples 1-3, where obtaining the plurality of measurable characteristics may include wirelessly receiving the current value for each of at least one of the plurality of measurable characteristics from a separate electronic device coupled to the user.

Example 11: The computer-implemented method of any one of Examples 1-3, where obtaining the current value for each of the plurality of measurable characteristics may include wirelessly receiving the current value for at least one of the plurality of measurable characteristics by way of a weather information application executing on the electronic device.

Example 12: The computer-implemented method of any one of Examples 1-3, where determining the temperature threshold may be further based on at least one of a demographic characteristic of the user or a medical condition of the user.

Example 13: The computer-implemented method of any one of Examples 1-3, where determining the temperature threshold may be further based on a characteristic of the electronic device.

Example 14: The computer-implemented method of Example 13, where the characteristic of the electronic device may include a heat flux of the electronic device.

Example 15: The computer-implemented method of Example 13, where the characteristic of the electronic device may include at least one of a location or an area of the electronic device in contact with the user.

Example 16: The computer-implemented method of Example 13, where the characteristic of the electronic device may include at least one of a current duration of usage of the electronic device by the user or a current use case of the electronic device.

Example 17: An electronic device may include (1) an enclosure with which a user is in physical contact, where the enclosure and the user are located within a local environment, (2) electronic circuitry within the enclosure, where the electronic circuitry includes at least one physical processor, and (3) physical memory including computer-executable instructions that, when executed by the physical processor, cause the electronic device to (a) obtain a current value for each of a plurality of measurable characteristics associated with at least one of the user or the local environment, (b) determine, based on the current value for each of the plurality of measurable characteristics, a temperature threshold for the electronic device, (c) measure a current temperature of the electronic device, (d) compare the current temperature to the temperature threshold, and (e) initiate, in response to the current temperature exceeding the temperature threshold, a heat mitigation operation of the electronic device to lower the current temperature.

Example 18: The electronic device of Example 17, where the electronic device may further include a wireless communication subsystem, where the electronic device obtains the current value for each of the plurality of measurable characteristics by wirelessly receiving, using the wireless communication subsystem, the current value of at least one of the plurality of measurable characteristics from a separate electronic device coupled to the user.

Example 19: The electronic device of either Example 17 or Example 18, where the electronic device may further include an ambient light sensor that measures at least one of an intensity of light or a bandwidth spectrum of light within the local environment.

Example 20: A non-transitory computer-readable medium that may include one or more executable instructions that, when executed by at least one processor of an electronic device, cause the electronic device to (1) obtain, within a local environment in which a user is in physical contact with the electronic device, a current value for each of a plurality of measurable characteristics associated with at least one of the user or the local environment, (2) determine, based on the current value for each of the plurality of measurable characteristics, a temperature threshold for the electronic device, (3) measure a current temperature of the electronic device, (4) compare the current temperature to the temperature threshold, and (5) initiate, in response to the current temperature exceeding the temperature threshold, a heat mitigation operation of the electronic device to lower the current temperature.

As detailed above, the computing devices and systems described and/or illustrated herein broadly represent any type or form of computing device or system capable of executing computer-readable instructions, such as those contained within the modules described herein. In their most basic configuration, these computing device(s) may each include at least one memory device and at least one physical processor.

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks.

In addition, one or more of the modules described herein may transform data, physical devices, and/or representations of physical devices from one form to another. For example, one or more of the modules recited herein may receive user, device, and/or local environment data to be transformed, transform the data to a dynamic temperature threshold, and use the result of the transformation to control the operation of the device to maintain a temperature of the device below the dynamic temperature threshold. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

In some embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A computer-implemented method comprising:
obtaining, within a local environment in which a user is in physical contact with an electronic device, a current value for each of a plurality of measurable characteristics associated with at least one of the user or the local environment;
determining, based on the current value for each of the plurality of measurable characteristics, a temperature threshold for the electronic device;
measuring a current temperature of the electronic device;
comparing the current temperature to the temperature threshold; and
initiating, in response to the current temperature exceeding the temperature threshold, a heat mitigation operation of the electronic device to lower the current temperature.

2. The computer-implemented method of claim 1, wherein the plurality of measurable characteristics comprises a characteristic of light detected within the local environment.

3. The computer-implemented method of claim 2, wherein the characteristic of the light detected within the local environment comprises at least one of an intensity of the light or a wavelength spectrum of the light.

4. The computer-implemented method of claim 1, wherein the plurality of measurable characteristics comprises a wind speed detected within the local environment.

5. The computer-implemented method of claim 1, wherein the plurality of measurable characteristics comprises a humidity detected within the local environment.

6. The computer-implemented method of claim 1, wherein the plurality of measurable characteristics comprises an ambient temperature detected within the local environment.

7. The computer-implemented method of claim 1, wherein the plurality of measurable characteristics comprises a heart rate of the user.

8. The computer-implemented method of claim 1, wherein the plurality of measurable characteristics comprises a respiration characteristic of the user.

9. The computer-implemented method of claim 1, wherein obtaining the current value for each of the plurality of measurable characteristics comprises measuring the current value for at least one of the plurality of measurable characteristics using a sensor of the electronic device.

10. The computer-implemented method of claim 1, wherein obtaining the plurality of measurable characteristics comprises wirelessly receiving the current value for each of at least one of the plurality of measurable characteristics from a separate electronic device coupled to the user.

11. The computer-implemented method of claim 1, wherein obtaining the current value for each of the plurality of measurable characteristics comprises wirelessly receiving the current value for at least one of the plurality of measurable characteristics by way of a weather information application executing on the electronic device.

12. The computer-implemented method of claim 1, wherein determining the temperature threshold is further based on at least one of a demographic characteristic of the user or a medical condition of the user.

13. The computer-implemented method of claim 1, wherein determining the temperature threshold is further based on a characteristic of the electronic device.

14. The computer-implemented method of claim 13, wherein the characteristic of the electronic device comprises a heat flux of the electronic device.

15. The computer-implemented method of claim 13, wherein the characteristic of the electronic device comprises at least one of a location or an area of the electronic device in contact with the user.

16. The computer-implemented method of claim 13, wherein the characteristic of the electronic device comprises at least one of a current duration of usage of the electronic device by the user or a current use case of the electronic device.

17. An electronic device comprising:
an enclosure with which a user is in physical contact, wherein the enclosure and the user are located within a local environment;
electronic circuitry within the enclosure, wherein the electronic circuitry comprises at least one physical processor; and
physical memory comprising computer-executable instructions that, when executed by the physical processor, cause the electronic device to:
obtain a current value for each of a plurality of measurable characteristics associated with at least one of the user or the local environment;
determine, based on the current value for each of the plurality of measurable characteristics, a temperature threshold for the electronic device;
measure a current temperature of the electronic device;
compare the current temperature to the temperature threshold; and
initiate, in response to the current temperature exceeding the temperature threshold, a heat mitigation operation of the electronic device to lower the current temperature.

18. The electronic device of claim 17, further comprising:
a wireless communication subsystem, wherein the electronic device obtains the current value for each of the plurality of measurable characteristics by wirelessly receiving, using the wireless communication subsystem, the current value of at least one of the plurality of measurable characteristics from a separate electronic device coupled to the user.

19. The electronic device of claim 17, further comprising:
an ambient light sensor that measures at least one of an intensity of light or a bandwidth spectrum of light within the local environment.

20. A non-transitory computer-readable medium comprising one or more executable instructions that, when executed by at least one processor of an electronic device, cause the electronic device to:
obtain, within a local environment in which a user is in physical contact with the electronic device, a current value for each of a plurality of measurable characteristics associated with at least one of the user or the local environment;
determine, based on the current value for each of the plurality of measurable characteristics, a temperature threshold for the electronic device;
measure a current temperature of the electronic device;
compare the current temperature to the temperature threshold; and
initiate, in response to the current temperature exceeding the temperature threshold, a heat mitigation operation of the electronic device to lower the current temperature.
